(19) United States
(12) Patent Application Publication
(10) Pub. No.: US 2022/0273172 A1
(43) Pub. Date: Sep. 1, 2022

(54) P SYSTEMS AND METHODS FOR DETECTING ATRIAL FIBRILLATION AND GENERATING NOTIFICATIONS THEREOF

(71) Applicant: Thomas M. Roe, Clarks Summit, PA (US)

(72) Inventor: Thomas M. Roe, Clarks Summit, PA (US)

(21) Appl. No.: 17/548,035

(22) Filed: Dec. 10, 2021

Related U.S. Application Data
(60) Provisional application No. 63/123,539, filed on Dec. 10, 2020.
(60) Provisional application No. 63/183,655, filed on May 4, 2021.

(51) Int. Cl. A61B 5/00; A61B 5/0245; A61B 5/283; G16H 10/60; G16H 20/10

(52) U.S. Cl. CPC A61B 5/0006; A61B 5/0022; A61B 5/02455; A61B 5/283; A61B 5/7405; A61B 5/742; A61B 5/7455; A61B 5/746; A61B 5/7465; A61B 5/7475; A61B 5/6869; G16H 10/60; G16H 20/10

(57) ABSTRACT

In one aspect, the disclosed technology relates to systems that provide real-time notification of the onset and offset of atrial fibrillation to a patient and a treating physician, enabling the physician to optimize and individualize the care for the AF patient. The care can include initiating or terminating anticoagulation medications, initiating or terminating other medications to treat cardiac conditions such as heart failure, coronary artery disease, etc. In another aspect, the disclosed technology relates to methods for detecting atrial fibrillation, and includes monitoring a patient for the onset of atrial fibrillation; and only upon detection of the onset of atrial fibrillation, and upon the direction of a treating physician, administering an anti-coagulant medication to the patient.

### CROSS REFERENCE TO RELATED APPLICATIONS

This application claims the benefit under 35 U.S.C. § 119(e) to U.S. provisional application No. 63/123,539, filed Dec. 10, 2020, and U.S. provisional application No. 63/183,655, filed May 4, 2021. The contents of each of these applications are incorporated by reference herein in their entirety.

### BACKGROUND

It is well documented that implantable cardiac monitors (ICM's) have a high degree of accuracy in detecting the development of atrial fibrillation (AF). Millions of patients currently have a permanent pacemaker, an implantable cardioverter defibrillator (ICD), or other type of ICM (the term “ICM,” as used herein, in intended to encompass pacemakers, ICD's, and other types of implantable cardiac monitors). Some of these patients have been diagnosed as having AF prior to the implantation of an ICM, and a significant number who do not yet have AF will develop it at some point during their lifetime. AF is documented as a major cause of stroke, and may result in the decompensation of chronic cardiac conditions such as heart failure and coronary artery disease. During atrial fibrillation, the upper chambers of the heart do not mechanically contract, permitting stasis of blood which may clot and subsequently travel to other areas of the cardiovascular system; additionally, the overall efficiency of cardiac performance is diminished.

Many patients experience very infrequent episodes of AF, and yet continue to take anticoagulant medications on an ongoing basis for the treatment of AF. While anticoagulant medications can be crucial to prevent stroke during episodes of AF, the use of such medications has been correlated directly to excessive bleeding events and are directly related to increases in patient morbidity and mortality. Many commonly used anticoagulant medications are costly, and may significantly impact a patient's lifestyle. Some anticoagulant medications necessitate dietary restrictions and alterations in medication usage, and require frequent blood tests.

Many patients with paroxysmal atrial fibrillation (PAF), or intermittent A-fib, currently “self-monitor” for the onset of AF. This is accomplished by various methods which include manually checking their pulse, or using devices such as pulse/blood-pressure monitors, APPLE WATCHES, KARDIA smart phone application, etc. Such “self-monitoring” patients are seeking individualized AF treatment. They may choose to use anticoagulant medications for limited time periods that commence with the onset of an AF event, and stop anticoagulation medications after sinus (normal) rhythm is restored. Atrial fibrillation does not result immediately in the development of clots; and the current standard of care for patients previously not on anticoagulant medication allows converting such patients out of AF by electrical shock if AF has been present for less than 48 hours in duration. If such a patient and his or her physician were quickly and reliably made aware of the onset of AF, the patient and physician could take action to reduce patient morbidity and mortality, and to reduce or eliminate the expense of required anti-coagulation medications.

Electronic medical records (EMRs) are the standard for documenting a patient's medical condition. EMRs can be programmed to alert a medical caretaker to important incoming medical information.

### SUMMARY

The disclosed technology is directed to providing real-time notification of the onset and offset of atrial fibrillation to a patient and a treating physician. This technology is a tool enabling the physician to optimize and individualize the care for the AF patient, and Applicant is unaware of any currently-available means to reliably provide individualized AF treatment. The care can include, without limitation, initiating or terminating anticoagulation medications, initiating or terminating other medications to treat cardiac conditions such as heart failure, coronary artery disease, etc. It would also be a means for the physician and patient to have heightened vigilance for clinical complications of atrial fibrillation.

The disclosed technology also is directed to methods for detecting atrial fibrillation. The methods include monitoring a patient for the onset of atrial fibrillation; and only upon detection of the onset of atrial fibrillation, and upon the direction of a treating physician, administering an anti-coagulant medication to the patient.

In another aspect of the disclosed technology, the methods also include directing the patient to alter medical therapy for the treatment of other cardiac conditions adversely effected by AF, heart failure, coronary artery disease, valvular heart disease, etc.

In another aspect, the disclosed technology can make the physician and patient more vigilant to monitor the patient's condition for deterioration due to the onset of AF.

In another aspect of the disclosed technology, the methods also include, at the direction of the treating physician, discontinuing the administration of the anticoagulant medication upon restoration of the cardiac sinus rhythm.

In another aspect of the disclosed technology, monitoring a patient for the onset of atrial fibrillation includes monitoring the patient for the onset of atrial fibrillation using an implantable medical device.

The disclosed technology also is directed to systems for detecting atrial fibrillation and generating a notification thereof. In one aspect, the systems include a sensing device having: one or more electrodes configured to sense electrical impulses representative of a heart rhythm; a controller communicatively coupled to the one or more electrodes, wherein the controller is configured to detect the occurrence of atrial fibrillation based on the outputs of the one or more electrodes, and to generate an output in response to the detection atrial fibrillation; and a transmitter communicatively coupled to the controller and configured to transmit a signal in response to receiving the output of the controller. The systems also include an alerting device communicatively coupled to the sensing device and configured to generate an alert in response to receiving the signal transmitted by the transmitter.

In another aspect of the disclosed technology, the alerting device includes a smart phone.

In another aspect of the disclosed technology, the alerting device further includes an application configured so that the application, when executed by the smartphone, facilitates communications between the smartphone and a communications network.

In another aspect of the disclosed technology, the application is further configured so that the application, when executed by the smart phone, causes the smart phone to generate and send over the communication network a notification regarding the onset or offset of atrial fibrillation.

In another aspect of the disclosed technology, the notification is in the form of at least one of an SMS text message, an e-mail, and a recorded message relayed via telephone.

In another aspect of the disclosed technology, the alerting device is at least one of a tablet, a notebook, a mini-computer, a micro-computer, a desktop computer, and an internet-enabled communication device.

In another aspect of the disclosed technology, the alerting device includes an application configured so that the application, when executed by the alerting device, facilitates communications between the alerting device and a communications network.

In another aspect of the disclosed technology, the application is further configured so that the application, when executed by the alerting device, causes the alerting device to generate and send over the communication network a notification regarding the onset or offset of atrial fibrillation.

In another aspect of the disclosed technology, the notification is in the form of at least one of an SMS text message, an e-mail, and a recorded message relayed via telephone.

In another aspect of the disclosed technology, the alerting device includes a BLUETOOTH® enabled device having an application configured to cause the BLUETOOTH® enabled device to generate the notification in the form of at least one of a text message, an e-mail, a phone call.

In another aspect of the disclosed technology, the alert includes a visual display.

In another aspect of the disclosed technology, the alert includes a sound.

In another aspect of the disclosed technology, the alert includes vibration of the receiving device.

In another aspect of the disclosed technology, the sensing device further includes a tone generator communicatively coupled to the controller; and the tone generator is configured to generate an audible tone in response to receiving the output of the controller.

In another aspect of the disclosed technology, the alerting device is communicatively coupled to a communication network; and the alerting device is further configured to generate, and transmit over the communication network, a notification in response to receiving the signal transmitted by the transmitter.

In another aspect of the disclosed technology, the alerting device is coupled to an electronic medical record (EMR).

In another aspect of the disclosed technology, a method for communicating the recurrence of AF, a first ever AF event, and/or an AF offset and return to Sinus Rhythm includes integrating an ICM or a wearable cardiac monitoring device and an EMR.

In another aspect of the disclosed technology, the development of atrial fibrillation can be entered in urgent, readily, and quickly identifiable EMR files, for example, positioned at the top of all incoming data and highlighted in red text.

In another aspect of the disclosed technology, the sensing device is configured to be implanted in the human body.

In another aspect of the disclosed technology, the sensing device is a wearable monitor capable of detecting atrial fibrillation.

In another aspect of the disclosed technology, the sensing device is an FDA-approved device.

In another aspect of the disclosed technology, the alerting device is further configured to maintain the alert until the alert is canceled or acknowledged by a user or a medical professional.

In another aspect of the disclosed technology, the controller is further configured to monitor the heart rhythm based on the outputs of the one or more electrodes.

In another aspect of the disclosed technology, the controller is further configured to generate a pattern of cardiac electrical activity in the form of an ECG wave based on the outputs of the one or more electrodes.

In another aspect of the disclosed technology, the controller is further configured to monitor and interpret the ECG wave; and to recognize characteristics in the ECG wave indicative of atrial fibrillation.

In another aspect of the disclosed technology, the altering device is further configured to generate another alert in response to a loss of communication between the alerting device and the communication network.

In another aspect of the disclosed technology, the altering device is further configured to generate another alert in response to a loss of communication, for a predesignated amount of time, between the alerting device and the another device over the communication network.

The disclosed technology also is directed to methods for detecting and notifying a patient and/or a medical professional of the occurrence of atrial fibrillation. In one aspect of the disclosed technology, the methods include providing a sensing device having: one or more electrodes configured to sense electrical impulses representative of a heart rhythm; a controller communicatively coupled to the one or more electrodes, wherein the controller is configured to detect the occurrence of atrial fibrillation based on the outputs of the one or more electrodes, and to generate an output in response to the detection atrial fibrillation; and a transmitter communicatively coupled to the controller and configured to transmit a signal in response to receiving the output of the controller. The methods also include providing an alerting device communicatively coupled to the sensing device and configured to generate an alert in response to receiving the signal transmitted by the transmitter; and administering a medication in response to the generation of the alert by the alerting device. The methods further include sensing the heart rhythm of a patient using the sensing device; and upon the detection of atrial fibrillation, generating the alert using the alerting device.

In another aspect of the disclosed technology, administering a medication in response to the generation of the alert by the alerting device includes administering an anticoagulant medication.

In another aspect of the disclosed technology, the methods further include implanting the sensing device in the patient.

In another aspect of the disclosed technology, the methods further include discontinuing administration of the medication upon re-establishment of cardiac sinus rhythm, and upon the direction of a physician.

In another aspect of the disclosed technology, the methods further include sending to a clinician, over a communication network, a notification of the detection of atrial fibrillation.

In another aspect of the disclosed technology, the methods further include directing the administration of the medication in response to receipt of the notification.

In another aspect of the disclosed technology, the methods further include sensing the heart rhythm based on the outputs of the one or more electrodes.

In another aspect of the disclosed technology, the methods further include generating a pattern of cardiac electrical activity in the form of an ECG wave based on the outputs of the one or more electrodes.

In another aspect of the disclosed technology, the methods further include monitoring and interpreting the ECG wave; and recognizing characteristics in the ECG wave indicative of atrial fibrillation.

In another aspect of the disclosed technology, the methods further include adjusting treatment or clinical awareness of cardiac conditions other than atrial fibrillation in response to the generation of the alert by the alerting device.

In another aspect of the disclosed technology, the methods further include adjusting treatment or clinical awareness of cardiac heart failure (CHF) and/or coronary artery disease in response to the generation of the alert.

### DESCRIPTION OF THE DRAWINGS

The following drawings are illustrative of particular embodiments of the present disclosure and do not limit the scope of the present disclosure. The drawings are not to scale and are intended for use in conjunction with the explanations provided herein. Embodiments of the present disclosure will hereinafter be described in conjunction with the appended drawings.

FIG. 1 is a schematic illustration of a system for detecting AF, and for generating an alert to notify the patient and/or a clinician of the onset of AF, and restoration of sinus rhythm.

FIG. 2 is a schematic illustration of a microcontroller unit of the system shown in FIG. 1.

FIG. 3 is a flow diagram of a method for detecting AF and generating notifications thereof.

### DETAILED DESCRIPTION

The inventive concepts are described with reference to the attached figures, wherein like reference numerals represent like parts and assemblies throughout the several views. The figures are not drawn to scale and are provided merely to illustrate the instant inventive concepts. The figures do not limit the scope of the present disclosure or the appended claims. Several aspects of the inventive concepts are described below with reference to example applications for illustration. It should be understood that numerous specific details, relationships, and methods are set forth to provide a full understanding of the inventive concepts. One having ordinary skill in the relevant art, however, will readily recognize that the inventive concepts can be practiced without one or more of the specific details or with other methods. In other instances, well-known structures or operation are not shown in detail to avoid obscuring the inventive concepts.

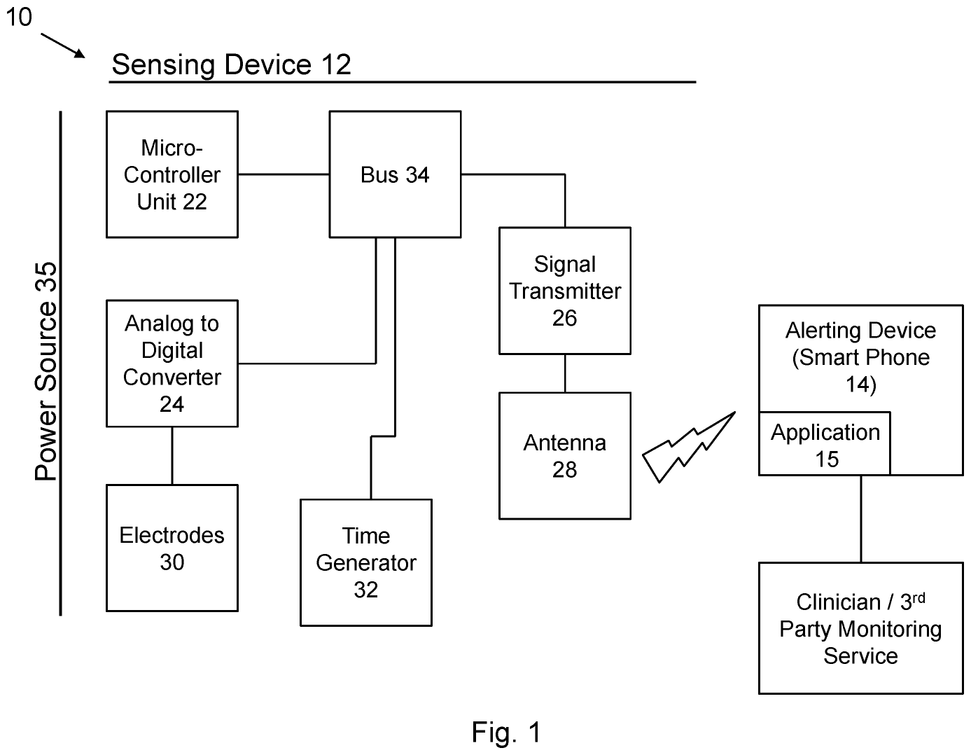

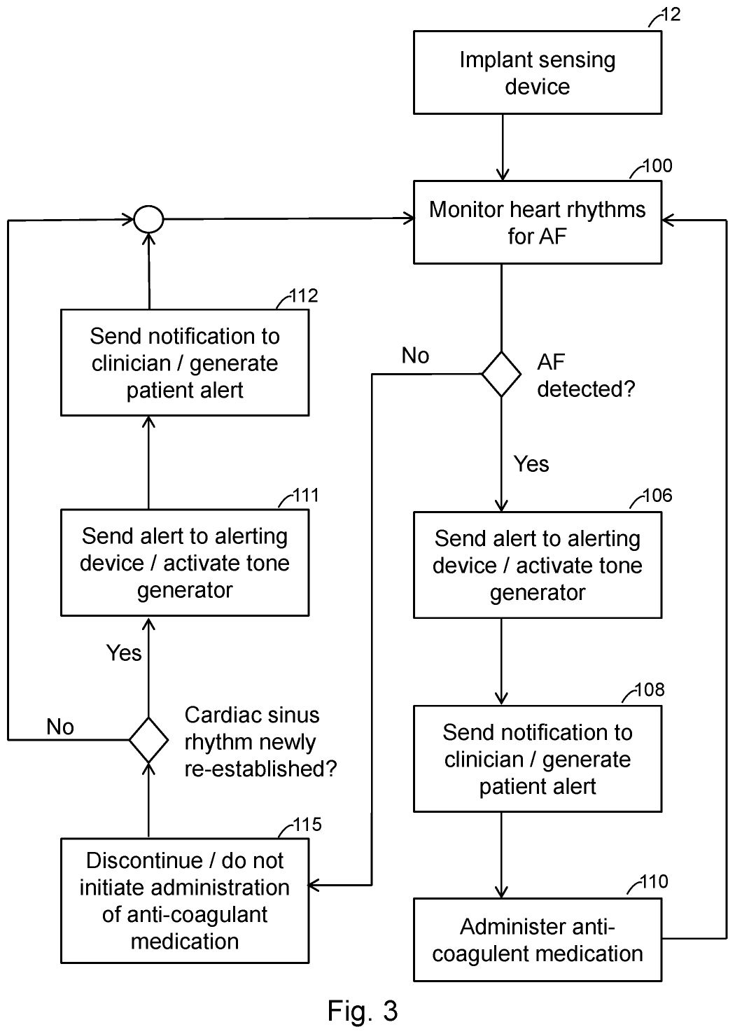

FIG. 1 depicts a system 10 for detecting AF, and for generating an alert to notify the patient and/or a clinician of the onset of AF. FIG. 3 depicts a method for detecting AF and generating notifications thereof.

The system 10 includes a sensing unit 12 and an alerting device. The sensing unit 12 is a type of implantable medical device. More specifically, the sensing unit 12 is configured to be implanted in a patient so that the sensing unit 12 can sense the heart rhythm of the patient, in a manner substantially similar or substantially identical to an ICM. In alternative embodiments, the sensing unit 12 can be a wearable device. The sensing device 12 is configured to monitor the heart rhythm, and to detect the onset of AF. Upon detecting the onset of AF, the sensing device 12, on a substantially real-time basis, generates and sends a signal to the alerting device. In response, the alerting device generates visual and audible alerts to notify the patient and/or a pre-designated clinician that AF has been detected. In one possible embodiment of the system 10, depicted in FIG. 1, the alerting device is a smart phone 14 equipped with an application 15 that causes the smart phone 14 to generate visual and audible alerts to notify the patient of the onset of AF. Also, the application 15 can be configured to cause the smart phone 14 to send an alert, and/or forward information to a pre-designated clinician, such as the patient's cardiologist, to notify the clinician that the patient is experiencing AF.

As shown schematically in FIG. 1, the sensing unit 12 comprises a housing (not shown); a microcontroller unit 22; an analog to digital converter 24; a signal transmitter 26; an antenna 28 electrically connected to the signal transmitter 26; two electrodes 30; a tone generator 32; an internal bus 34 that facilitates communication between the various electrical components of the sensing unit 22; and a power source 35. The power source 35 can be, for example, a battery capable of being recharged on a wireless basis by inductive coupling or another suitable means.

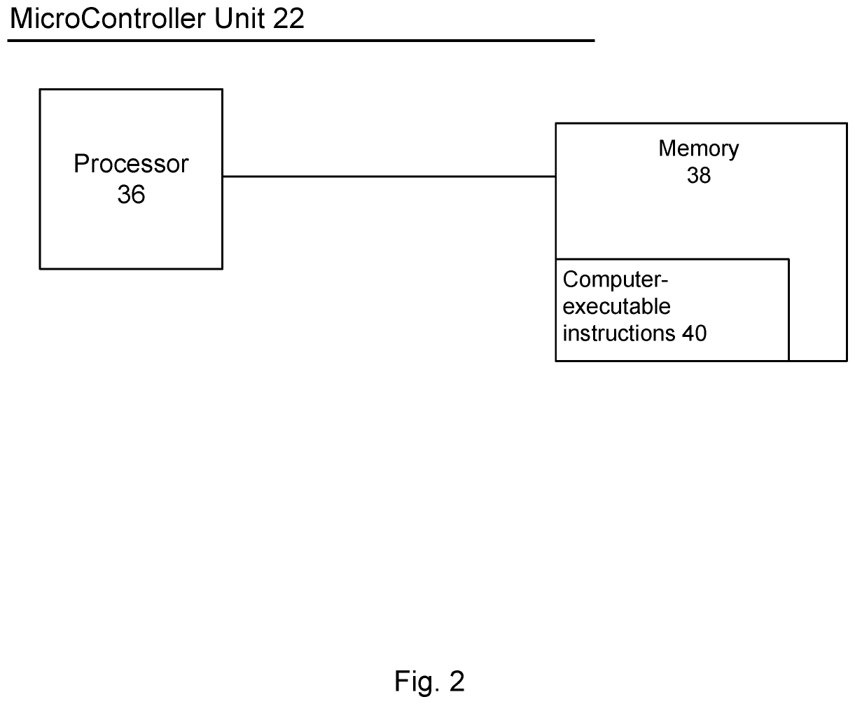

Referring to FIG. 2, the microcontroller unit 22 comprises a processor 36, such as a microprocessor; a memory 38 communicatively coupled to the processor 36; and computer executable instructions 40 stored on the memory 38. The microcontroller unit 22 can include other components, the disclosure of which is not necessary to an understanding of the disclosed technology.

The electrodes 30 sense the electrical signals that are sent to the patient's heart by the autonomic nervous system, to cause the heart to contract. The electrodes 30 are communicatively coupled to the analog to digital converter 24. The analog to digital converter 24 converts the analog signal output by each electrode 30 to a digital signal and sends the digital signal to the processor 36 by way of the internal bus 34.

The computer executable instruction 40, upon being executed by the processor 36, cause the processor 36 to generate a pattern representing electrical activity of the heart in the form of an ECG wave, based on the signals received from the analog to digital converter 24. The processor 36 continually monitors and interprets the ECG wave; and can recognize characteristics in the ECG wave indicative of AF using, without limitation, conventional techniques known to those skilled in the art of cardiac monitoring (item 100 in FIG. 3).

The processor 36 is communicatively coupled to the tone generator 32 and the signal transmitter 26 by way of the internal bus 34. Upon detecting the onset of AF, the processor 36 generates an output that, when received by the tone generator 32, causes the tone generator 32 to generate an audible alert to notify the patient of the onset of AF (block 106 of FIG. 3). Also, the processor 36 generates a second output that, when received by the signal transmitter 26, causes the signal transmitter 26 to generate a signal that is transmitted to smart phone 14 by way of the antenna 28 (block 106 of FIG. 3). The signal transmitter 26 can communicate with the smart phone 14 via RF transmission, such as BLUETOOTH® or by other suitable means.

The sensing device 12 can be configured to generate the above alerts and outputs indicating the occurrence of AF after a pre-set amount of time has elapsed following detection of the onset of AF. The system 10 can be configured so that the pre-set amount of time can be varied, and can be input or set by the clinician.

The smart phone 14, executing the application 15, interprets the alert signal received from the sensing device 12 as an indication that the patient is experiencing AF. In response, the smart phone 14 displays, on a real-time or near real-time basis, a visual notification stating that the patient is experiencing AF. In addition, the application 15 can give the user options to enable the smart phone 14 to generate an audible alert and/or to vibrate upon receiving the incoming signal from the sensing device 12, to further alert the patient of the onset of AF (block 108 of FIG. 3). The smart phone 14 can be configured so that the alerts cannot be canceled until the patient (or a designated caregiver) acknowledges the alerts.

The smart phone 14, executing the application 15, sends an alert to the pre-designated clinician on a real-time or near real-time basis, notifying the clinician that the patient is experiencing AF (block 108). The notification can be sent as an e-mail, SMS text message, pre-recorded voice recording, electronic medical record, or other suitable means, over a suitable telecommunications network. If desired, other recipients for the alerts can be designated. For example, the alerts can be sent to a designated caretaker or family member in situations where the patient is physically or mentally incompetent. The alerts also can be sent, for example, to a designated third-party monitoring center or alarm monitoring service.

The patient, upon receiving the indications that he or she is experiencing AF, can contact the patient's physician or other clinician so that the clinician can evaluate the patient's condition, and if necessary prescribe treatment such as anticoagulant medication (block 110 of FIG. 3). Also the physician can adjust treatment or clinical awareness of other cardiac conditions such as cardiac heart failure (CHF), coronary artery disease, valvular heart disease, etc.; and patient and physician vigilance for deterioration of the patient's condition due to AF can be increased upon receiving the notification of AF. Alternatively, because the notification of AF also is sent to the clinician, the contact between the patient and clinician can be initiated by the clinician instead of the patient.

The sensing device 12 can be further configured to recognize when the patient's natural sinus rhythm has been re-established, and to generate a corresponding alert that can be sent to the clinician by way of the smart phone 14. The alert can be generated after a pre-set amount of time has elapsed following the re-establishment of natural sinus rhythm. The system 10 can be configured so that the pre-set amount of time can be varied, and can be input or set by the clinician. The clinician, e.g., the patient's treating cardiologist or other physician, or a third party monitoring agency at this point can re-evaluate the condition of the patient, and if appropriate, discontinue administration the anticoagulant medication (blocks 111, 112 of FIG. 3). Also, the treating physician can direct withdrawal of therapies and normalization of patient surveillance (monitoring) upon restoration of the natural sinus rhythm.

The system 10 thus can provide the clinician with a real time or near real-time indication of when treatment for AF is needed, and at least implicitly, when such treatment is not needed. The ability to quickly and reliably alert the clinician to the onset of AF can eliminate the need to medicate the patient for AF when AF is not present and a physician has directed that medication is not required at the time.

Thus, because AF medication is administered only when needed, the patient is not subjected unnecessarily to the side effects, expense, and other negative aspects of such medication. Also, in situations where the patient is being medicated for AF on a continuous basis, the system 10 can provide the clinician with an indication of AF and other adverse events that nevertheless may occur even though the patient is being medicated. For example, the decompensation of heart failure, coronary artery ischemia, and other adverse cardiac events do occur in some patients when atrial fibrillation occurs. It is contemplated that the use of the system 10 in the above manner potentially can lead to recognition by the U.S. Food and Drug Administration of a novel process of PAF treatment, and can open an entirely new indication for ICM-type implants in patients who otherwise do not require an IMD such as a pacemaker or ICD. It is believed that patients with documented PAF, patients identified as being at risk for PAF (and there are millions), patients that have suffered cryptogenic cerebrovascular accident (CVA), heart failure patients who experience a decompensation of their condition, coronary artery disease patients who may experience increase in coronary ischemia, and others could benefit from the use of the system 10 and variants thereof. The limited and targeted anticoagulated therapy made available through the use of the system 10 and its variants has the potential to increase patient safety; produce better cardiovascular outcomes, such as decreased decompensated heart failure, decreased ischemic events, etc.; and reduce medication use along with the expense and side effects associated therewith.

In addition to transmitting alerts, the sensing device 12 can be configured to transmit to the smart phone 14 information such as the raw digital data generated by the electrodes 30 and the analog to digital converter 24, and/or the ECG wave generated by the microcontroller unit 22 based on the raw data. Data transmission can be continuous, or can be initiated upon the onset of AF. The microcontroller 22 can be configured to maintain a cache of the data in the memory 38, and to send the cached information to the smart phone 14 upon the detection of an AF event, to provide a history of cardiac function preceding the onset of AF. Monitoring devices can provide rhythm strips, ECGs that can include a short segment typically one minute prior to AF onset with the strip continuous into the AF event, and a similar approximately one minute strip documenting the termination of AF and the restoration of sinus rhythm. The duration of one minute, or just 15 seconds, etc. is variable depending on the manufacturer, and can be programmed to provide for different clinical situations.

The smart phone 14, executing the application 15, can store the information received from the sensing device 12, so that the information can be sent to the clinician at a later time to assist the clinician in evaluating the AF event. The information potentially can provide the clinician with insight into, for example, the duration and severity of the AF event, the factors that may have given rise to the AF event, ventricular heart rates while in atrial fibrillation, etc. Alternatively, the smart phone 15 can be configured to re-transmit the information to the clinician immediately upon receipt from the sensing device 12.

The altering device 14 can be further configured to generate an alert when the altering device 14 is unable to communicate over the telecommunication network, e.g., when the alerting device 14 is unable to send alerts to the pre-designated clinician due to network issues. To help avoid nuisance alerts, the alert can be generated after communication has been lost for a predesignated amount of time.

In alternative embodiments, the alerting device can be a device other than the smart phone 14. In particular, the alerting device can be virtually any telecommunication device capable of relaying information between the sensing device 10 and a telecommunication network. For example, in alternative embodiments of the system 10, the altering device can be a portable computer including, but not limited to, a tablet, a notebook, a mini-computer, a micro-computer, a desktop computer, etc.

In other alternative embodiments, the alerting device, e.g., the smart phone 14, can be configured to perform the processing operations performed by the sensing device 12 in the system 10. More specifically, the sensing device 12 can be configured to continually stream to the smart phone 14 the raw cardiac data generated by the electrodes 30 and the analog to digital converter 24. The smart phone 14 can be configured to process the raw data and generate the ECG wave; continually monitor and interpret the ECG wave and recognize characteristics in the ECG wave indicative of AF; and initiate the alerting sequence upon detecting the onset of AF.

We claim:

1. A system for detecting atrial fibrillation and generating a notification thereof, comprising:
a sensing device comprising: one or more electrodes configured to sense electrical impulses representative of a heart rhythm; a controller communicatively coupled to the one or more electrodes, wherein the controller is configured to detect the occurrence of atrial fibrillation based on the outputs of the one or more electrodes, and to generate an output in response to the detection atrial fibrillation; and a transmitter communicatively coupled to the controller and configured to transmit a signal in response to receiving the output of the controller; and
an alerting device communicatively coupled to the sensing device and configured to generate an alert in response to receiving the signal transmitted by the transmitter.

2. The system of claim 1, wherein the alerting device comprises a smart phone.

3. The system of claim 2, wherein the alerting device further comprises an application configured so that the application, when executed by the smart phone, facilitates communications between the smart phone and a communications network.

4. The system of claim 3, wherein the application is further configured so that the application, when executed by the smart phone, causes the smart phone to generate and send over the communication network a notification regarding the onset or offset of atrial fibrillation.

5. The system of claim 4, wherein the notification is in the form of at least one of an SMS text message, an e-mail, and a recorded message relayed via telephone.

5. The system of claim 1, wherein the alert comprises a visual display.

6. The system of claim 1, wherein the alert comprises a sound.

7. The system of claim 1, wherein the alert comprises vibration of the receiving device.

8. The system of claim 1, wherein: the sensing device further comprises a tone generator communicatively coupled to the controller; and the tone generator is configured to generate an audible tone in response to receiving the output of the controller.

9. The system of claim 1, wherein: the alerting device is communicatively coupled to a communication network; and the alerting device is further configured to generate, and transmit over the communication network, a notification in response to receiving the signal transmitted by the transmitter.

10. The system of claim 1, wherein the alerting device is communicatively coupled to an electronic medical record.

11. The system of claim 10, wherein the electronic medical record is configured to create a highlighted quickly and readily identifiable file in the patient's chart.

12. The system of claim 9, wherein the alerting device is communicatively coupled to an electronic medical record.

13. The system of claim 12, wherein the electronic medical record is configured to create a highlighted quickly and readily identifiable file in the patient's chart in response to the notification.

14. The system of claim 1, wherein the sensing device is configured to be implanted in the human body.

15. The system of claim 1, wherein the sensing device is configured as a wearable monitor.

16. The system of claim 1, wherein the alerting device is further configured to maintain the alert until the alert is canceled by a user or a medical professional.

17. The system of claim 1, wherein the controller is further configured to monitor the heart rhythm based on the outputs of the one or more electrodes.

18. The system of claim 17, wherein the controller is further configured to generate a pattern of cardiac electrical activity in the form of an ECG wave based on the outputs of the one or more electrodes.

19. The system of claim 18, wherein the controller is further configured to monitor and interpret the ECG wave; and to recognize characteristics in the ECG wave indicative of atrial fibrillation.

20. The system of claim 1, wherein the alerting device is at least one of a tablet, a notebook, a mini-computer, a micro-computer, a desktop computer, and an internet-enabled communication device.

21. The system of claim 20, wherein the alerting device comprises an application configured so that the application, when executed by the alerting device, facilitates communications between the alerting device and a communications network.

22. The system of claim 21, wherein the application is further configured so that the application, when executed by the alerting device, causes the alerting device to generate and send over the communication network a notification regarding the onset or offset of atrial fibrillation.

23. The system of claim 22, wherein the notification is in the form of at least one of an SMS text message, an e-mail, and a recorded message relayed via telephone.

24. The system of claim 1, wherein the altering device is further configured to generate another alert in response to a loss of communication between the alerting device and the communication network.

25. A method for detecting atrial fibrillation and generating notifications thereof, comprising:
providing a sensing device comprising: one or more electrodes configured to sense electrical impulses representative of a heart rhythm; a controller communicatively coupled to the one or more electrodes, wherein the controller is configured to detect the occurrence of atrial fibrillation based on the outputs of the one or more electrodes, and to generate an output in response to the detection atrial fibrillation; and a transmitter communicatively coupled to the controller and configured to transmit a signal in response to receiving the output of the controller;
providing an alerting device communicatively coupled to the sensing device and configured to generate an alert in response to receiving the signal transmitted by the transmitter;
sensing the heart rhythm of a patient using the sensing device; and
upon the detection of atrial fibrillation, generating the alert using the alerting device.

26. The method of claim 25, further comprising administering a medication in response to the generation of the alert by the alerting device.

27. The method of claim 26, wherein administering a medication in response to the generation of the alert by the alerting device comprises administering an anticoagulant medication.

28. The method of claim 25, further comprising implanting the sensing device in the patient,

29. The method of claim 26, further comprising discontinuing administration of the medication upon re-establishment of cardiac sinus rhythm.

30. The method of claim 25, wherein:
the controller is further configured to generate a second output in response to the re-establishment of cardiac sinus rhythm;
the transmitter is further configured to transmit a second signal in response to receiving the second output of the controller; and
the alerting device is further configured to generate an alert in response to receiving the second signal transmitted by the transmitter.

31. The method of claim 26, further comprising sending to a clinician, over a communication network, a notification of the detection of atrial fibrillation.

32. The method of claim 31, further comprising directing the administration of the medication in response to receipt of the notification.

33. The method of claim 25, further comprising sensing the heart rhythm based on the outputs of the one or more electrodes.

34. The method of claim 33, further comprising generating a pattern of cardiac electrical activity in the form of an ECG wave based on the outputs of the one or more electrodes.

35. The method of claim 34, further comprising monitoring and interpreting the ECG wave; and recognizing characteristics in the ECG wave indicative of atrial fibrillation.

36. A method for treating atrial fibrillation, comprising; monitoring a patient for the onset of atrial fibrillation; and only upon detection of the onset of atrial fibrillation, directing the administration of an anti-coagulant medication to the patient.

37. The method of claim 36, further comprising directing discontinuation of the administration of the anticoagulant medication upon restoration of the cardiac sinus rhythm.

38. The method of claim 36, wherein monitoring a patient for the onset of atrial fibrillation comprises monitoring the patient for the onset of atrial fibrillation using an implantable medical device.

39. The method of claim 25, further comprising adjusting treatment or clinical awareness of cardiac conditions other than atrial fibrillation in response to the generation of the alert by the alerting device.

40. The method of claim 39, wherein adjusting treatment or clinical awareness of cardiac conditions other than atrial fibrillation in response to the generation of the alert by the alerting device comprises adjusting treatment or clinical awareness of cardiac heart failure (CHF) and/or coronary artery disease.
